(19) United States
(12) Patent Application Publication
(10) Pub. No.: US 2003/0108845 A1
(43) Pub. Date: Jun. 12, 2003

(54) Framework assembly for providing a passive fit with divergent implants

(76) Inventors: Paul L. Giovannone, Barneveld, NY (US); Robert D. Hicks, Fort Gratiot, MI (US)

Correspondence Address: HESLIN ROTHENBERG FARLEY & MESITI PC, 5 COLUMBIA CIRCLE, ALBANY, NY, 12203, US

(21) Appl. No.: 10/013,622

(22) Filed: Dec. 11, 2001

(51) Int. Cl. A61F 2/28; A61C 8/00

(52) U.S. Cl. 433/173; 623/17.17; 623/16.11; 623/901

(57) ABSTRACT

A framework assembly that supports a prosthesis and passively fits onto implants embedded in a bone. The framework assembly comprises a first abutment attachable to a first implant and a second abutment attachable to a second implant. The first abutment is attachable to the second abutment by a joint which causes the first abutment to be moveable relative to the second abutment. The framework assembly may also include additional abutments connected to the first and second abutments by either a rigid connecting segment or another joint.

### FIELD OF THE INVENTION

The present invention generally relates to a framework assembly for passively attaching to divergent implants in a person's jaw bone and for supporting dentures, although the framework assembly of the present invention may be attached to implants inserted in bones throughout the body to support a wide variety of prosthesis.

### BACKGROUND OF THE INVENTION

In conventional dental implant therapy, root form, endosseous implants are surgically inserted below the periosteum into either the maxilla or mandible for supporting denture prosthesis to restore comfortable and efficient oral function, as well as to provide acceptable aesthetic replacement of missing teeth.

The implants are installed in bores formed in the maxilla or mandible. During insertion, the gum tissue overlying the area of the maxilla or mandible in which the implant is to be affixed is cut and folded back to expose the bone surface. Cylindrical bores are then formed in the bone for the insertion of the implant. Each implant typically includes a threaded bore opening out of the bone tissue for attaching the denture prosthesis and a generally cylindrical body having an outer periphery surface which slides frictionally along, or screws into, the bores. Once pushed or screwed in, the implants are snugly held within the bore and. An extension may also be provided to extend through the tissue from the bone.

The implants, over time, become integrated with the bone structure into which they are embedded with concurrent formation of new bone through a natural body occurrence called osteogenesis. After a prescribed healing period of about three to six months, the implants can then be used as support or anchor points for dental prosthetic devices such as dentures.

One common denture prosthesis used with implant therapy includes a metal substructure or framework which attaches to the implants and supports the dentures. This substructure or framework is fastened to the dental implants using a series of metal retaining screws, tightened to predetermined torque specifications.

The fabrication of conventional frameworks involves a variety of meticulous clinical and laboratory steps to assure that it interfaces properly with existing implants. Current fabrication procedures and materials used by the dental laboratory place uncontrollable limits on the ability of a laboratory staff to produce frameworks which consistently seat passively to existing implant. Some of the steps involved include identifying the most accurate materials, impression making, master cast fabrication, laboratory wax-up, investing, casting and finishing. Each of these steps could be a source of potential distortion that can result in poor fitting or non-passive seating.

The most popular framework used today is made by casting the entire framework as one piece from a precious metal such as, for example, gold. These cast frameworks are, however, expensive, rigid, non-forgiving, and cannot accommodate the micro-inaccuracies caused during fabrication, or the micro-movements of the implants, or the flexure of the mandible.

In order to accurately and passively fit all of the implants, the single piece framework must be cast to small tolerances in order to attach to multiple implants in a person's mouth which may be angled in more than one direction. However, distortions incurred during fabrication make this procedure extremely difficult. The cast bar framework is limited to the lengths and angles of its sections after casting.

Another problem with conventional rigid dental frameworks is that the interface between the framework and implants cannot adapt to the altering configurations necessitated by the structure of a patient's mouth. Since the implants are embedded surgically in the jaw bone of the patient, the implants may be angled inwardly or outwardly from the jaw bone depending upon a patient's jaw bone configuration. This can be problematical, especially when the implant is embedded in the anterior portion of a patient's mouth in which case the implant may be at a severe outward angle. A misaligned implant is often divergent from other implants or teeth in the mouth making the fabrication of a rigid framework which fits passively at all locations with the implants impossible.

There are many factors which cause the implants to become divergent, positional distorted or move away from their originally specified referenced positions. For example, if the metal framework does not fit accurately onto the dental implants, it places an undesirable load on the denture system causing biological and prosthetic complications. When a dental implant is overloaded or a lateral load is placed on it due to non-passive seating of the framework, the integration between implant and bone can be compromised. This can result in devastating failure of the prosthetic restoration and other subsequent maladies may occur including, for example, compromised periodontal health, crestal bone loss around implants and loss of osseointegration, and poor nutrition and psychological wellbeing.

A number of methods and procedures currently exist to verify positional accuracy of the implants to the framework in the mouth at various stages of the fabrication procedure. Some of these procedures include, for example, implant master cast verification, sectioning and soldering procedures by casting a fixed detachable hybrid prosthesis into multiple segments and the soldering together, luting the implant framework or definitive prosthesis to implant components intraorally, laser welding of titanium and electric-discharge machining. A more detailed discussion of these conventional procedures is provided by Alvin G. Wee BDS, MS, Steven A. Aquilino, DDS, MS, and Robert L. Schneider, DDS, MS, “Strategies to Achieve Fit in Implant Prosthodontics: A Review of the Literature,” The International Journal of Prosthodontics, Vol. 12, No. 2, p. 167, 1999, which is hereby incorporated herein by reference. All of these frameworks and procedures to make the frameworks, however, involve a rigid, non-forgiving framework to be inserted into a patient's mouth.

Despite the use of these new procedures, many factors still influence the accuracy of the fit and problems continue to exist in achieving a passive fit for divergent implants. These problems continue to be caused by, for example, inaccuracy of the individual components, implant machining tolerances, and flexure of the mandible. Thus, an exact replica of the implants is extremely difficult, if not impossible, to realize and some patients are not presently amenable to the installation of permanent dental prostheses.

Moreover, conventional techniques will often stack many different metal groups on top of one another to support the dental restoration. The mixing of materials can promote corrosion due to galvanic reactions between the different alloys used. Titanium or a titanium alloy is the preferred material to use in dental surgery because it is more compatible with bone and less expensive than the precious metals, such as gold, used. However, it is extremely difficult to cast titanium or a titanium alloy.

### SUMMARY OF THE INVENTION

The shortcomings of the prior art may be alleviated by using a framework assembly in accordance with one or more principles of the present invention. The framework assembly of the present invention may be attachable to implants embedded in a wide variety of bones throughout the body to support many different types of prosthesis such as, for example, dentures, eyes, ears, nose or the like. Additionally, other uses may be made of the invention which fall within the scope of the claimed invention but which are not specifically described below.

In one aspect of the invention, there is provided a framework assembly for supporting a prosthesis and for passively fitting onto implants installed in a bone. The framework assembly comprises a first abutment attachable to a first implant and a second abutment attachable to a second implant. The first abutment is attachable to the second abutment by a joint and is movable relative to the second abutment.

In another aspect of the invention, there is provided a method of assembling a framework for supporting a prosthesis and for passively fitting onto implants installed in a bone. The method comprises providing at least two abutments, a connecting segment having a male member connected to one end of the connecting segment, and a socket housing corresponding to adjacent pairs of abutments. The socket housing includes an upper portion and a lower portion. The upper and lower portions of the socket housing have corresponding recesses that define a socket when joined. The method further comprises laser welding either the upper portion or the lower portion of the socket housing to one of the abutments and inserting the male member into the recess formed by the upper or lower portion of the socket housing laser welded to one of the abutments. The method further comprises laser welding the other of the upper portion or lower portion of the socket housing together with the male member in the socket to form a joint and laser welding the other end of the connecting segment opposite the male member to the other of the adjacent abutments.

A framework assembly constructed in accordance with the principles of the present invention has certain features which permit attachment to divergent implants that rigid, non-forgiving, cast frameworks are unable to accommodate. One of these features includes a joint enabling adjacent abutments to move (e.g. rotate, swivel, articulatable or slide) relative to each other in order to compensate for the micro-movement or micro-inaccuracies that may occur from the impression techniques of the doctor down through the fabrication techniques of the laboratory, in addition to the flexure and movement of the jaw bone over time.

Another feature includes the use of laser welding techniques to fabricate the framework which avoids any waxing, casting, finishing and soldering and eliminates the need to use expensive precious metal alloys such as palladium, platinum and gold.

The framework described and claimed herein assures a passive fit between the framework and the implants inserted in bone.

Additional features and advantages are realized through the techniques of the present invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 illustrates a perspective view of the assembled framework constructed in accordance with the principles of the present invention connected to implants inserted in a jaw bone;

FIG. 2 illustrates an exploded view of the framework shown in FIG. 1;

FIG. 3 illustrates a partial perspective view of the male member of a connecting segment inserted into the curved recess defined in one portion of the socket housing;

FIG. 4 illustrates a side view of the joint constructed in accordance with the principles of the present invention demonstrating movement in two dimensions;

FIG. 5 illustrates a top view of the joint shown in FIG. 4 demonstrating movement in a third dimension;

FIG. 6 illustrates a perspective view of another embodiment of the assembled framework constructed in accordance with the principles of the present invention connected to implants inserted in a jaw bone;

FIG. 7 illustrates a cross-sectional view of an embodiment of the joint constructed in accordance with the principles of the present invention showing the beveled surface of the cylindrical shaft of the connecting segment and the end wall of socket housing;

FIG. 8 illustrates a perspective view of the dual purpose impression posts used to form abutments constructed in accordance with the principles of the present invention; and

FIG. 9 illustrates a perspective view of one type of denture prosthesis having clips and O-ring attachments for attaching to a framework constructed in accordance with the principles of the present invention.

### DETAILED DESCRIPTION OF EMBODIMENTS OF THE INVENTION

Presented herein is an assembly of prefabricated, machined components into a prosthetic framework that is joined by means of a laser welding process. This framework allows passive seating on non-parallel or divergent implant fixtures by enabling adjacent abutments to move relative to each other in order to compensate for the micro-movement or micro-inaccuracies that occur from the impression techniques of the doctor down through the fabrication techniques of the laboratory, as well as for the flexure and movement of the jaw bone.

The framework assembly constructed in accordance with the principles of the present invention provides metal alloy components made from, preferably, titanium or titanium alloy because of its resistance to corrosion, and assembled by laser-welding procedures. As will be discussed in more detail below, the components of the framework assembly are capable of moving in multiple directions relative to each other in order to adjust between multiple implants, while addressing the problems associated with divergent implant fixtures. This framework also avoids the distortions occurring during the waxing, casting and torch soldering stages of conventional fabrication procedures.

For simplicity reasons only, the framework assembly will be discussed with reference to dentistry, particularly for attaching to dental implants and for supporting dentures. Although it should be understanding that the framework assembly may be used to passively mount on implants embedded in many other supporting bones throughout the body to support other types of prosthesis, such as, for example, ears, eyes, nose, or the like.

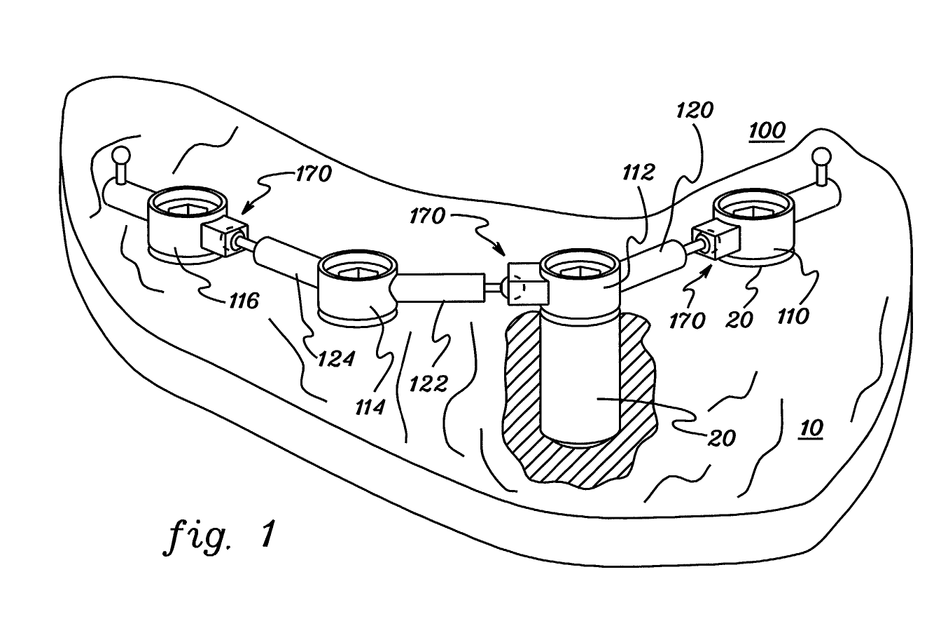

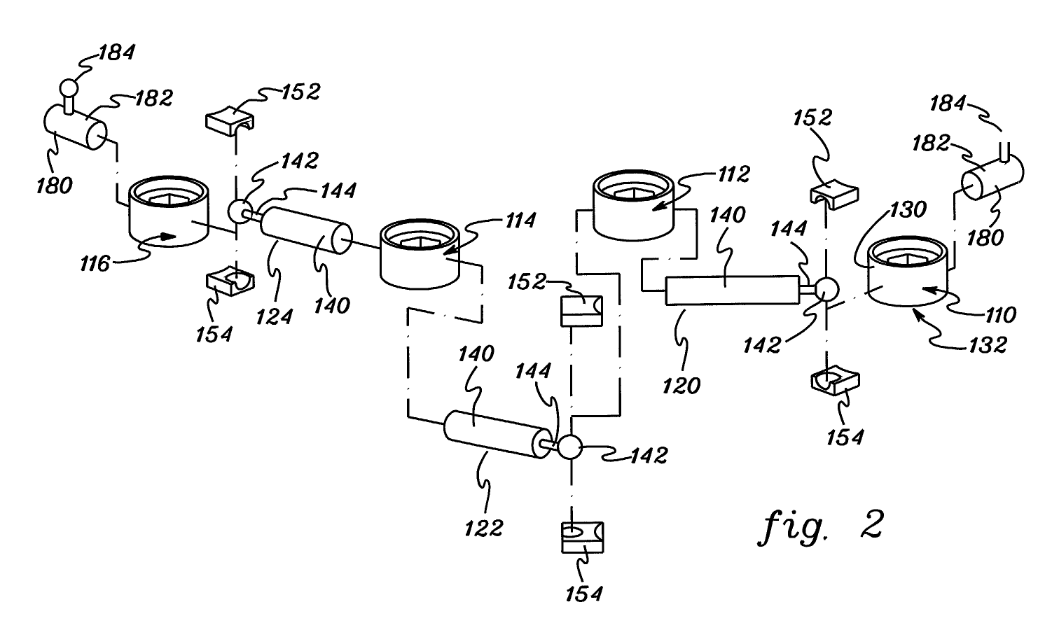

Turning now to the illustrative embodiment shown in FIGS. 1 and 2, a framework assembly 100 is attached to implants 20 installed in the maxilla or mandible 10 of a patient's mouth. Framework assembly 100 includes a plurality of abutments 110, 112, 114, 116 corresponding to the number of implants 20 existing in the patient's mouth and a plurality of connecting segments 120, 122, 124 secured between adjacent pairs of abutments 110 and 112, 112 and 114, 114 and 116, respectively. The abutments 110, 112, 114, 116 and connecting segments 120, 122, 124 are preferably milled out of medical grade titanium or titanium alloy, although other biocompatible metals, composites, plastics or the like may be used.

Each abutment 110, 112, 114, 116 of framework assembly 100 includes a cylindrical tube 130 having an annular seat 132 in the lower end of the tube corresponding to, or for receiving, the top portion of implant 20 inserted in a patient's bone. Cylindrical tube 130 defines a through bore in registry with the threaded bore of implant 20. An abutment retaining screw or threaded member is received in each through bore and threaded into the threaded bore of implant 20 to secure the abutment to the dental implant.

Segments 120, 122, 124 connecting adjacent pairs of abutments are sized to close proximity to the distances between adjacent pairs of abutments. A first segment 120 extending between abutments 110 and 112 includes an elongated cylindrical shaft 140, although other shapes may be used, connected at one end to a male member 142 such as, for example, a convex spherical or ball member, by a cylindrical extension 144 having a smaller diameter than cylindrical shaft 140. The end of cylindrical shaft 140 opposite to male member 142 is attachable by laser welding means to abutment 112 as discussed in more detail below.

Abutment 110 includes a socket housing 150 extending radially from the outer surface of cylindrical tube 130 forming abutment 110 towards adjacent abutment 112. Socket housing 150 includes an upper portion 152 and a lower portion 154, each defining a curved recess 156, 158, respectively, for accommodating a portion of male member 142.

As shown in FIG. 3, upper portion 152 and lower portion 154 of socket housing 150 each includes an outer side wall 160 and an opposed inner side wall 162 joined by spaced apart end walls 164, 166, a top wall 168 and a bottom wall 170. End wall 164 is formed to fit intimately with the outer surface of the abutments. In an alternate embodiment, upper and lower portions 152, 154 may have a curved outer surface as the top, bottom and outer sides to form a cylindrical outer surface. With use in the dental field, the curved outer surfaces for the upper and lower portions may be preferred to avoid sharp corners and edges in a patient's mouth.

Curved recess 156, 158 are provided in inner side wall 162 and end wall 166 of each portion 152, 154. When upper portion 152 and lower portion 154 of socket housing 150 are joined together with respective inner side walls 162 contacting each other, the respective curved recesses 156, 158 are joined to thereby form a socket for receiving at least a portion of male member 142.

The socket, or alternatively, the female member, formed by the union of upper and lower portions 152, 154 of socket housing 150 defines a generally hollow concave configuration having a surface portion that may correspond to or complement male member 142. The socket includes an open slot or port formed through end walls 166 of upper and lower portion 152, 154, respectively, for access or insertion of a portion of male member 142 and/or cylindrical extension 144 of connecting segment 144.

Male member 142 is configured to move (e.g rotate, swivel, articulate and/or slide) within the hollow interior while being confined therein. Once male member 142 is captured within the hollow interior of the socket, upper and lower portion 152, 154 comprising socket housing 150 are secured together by laser welding means, as discussed in more detail below.

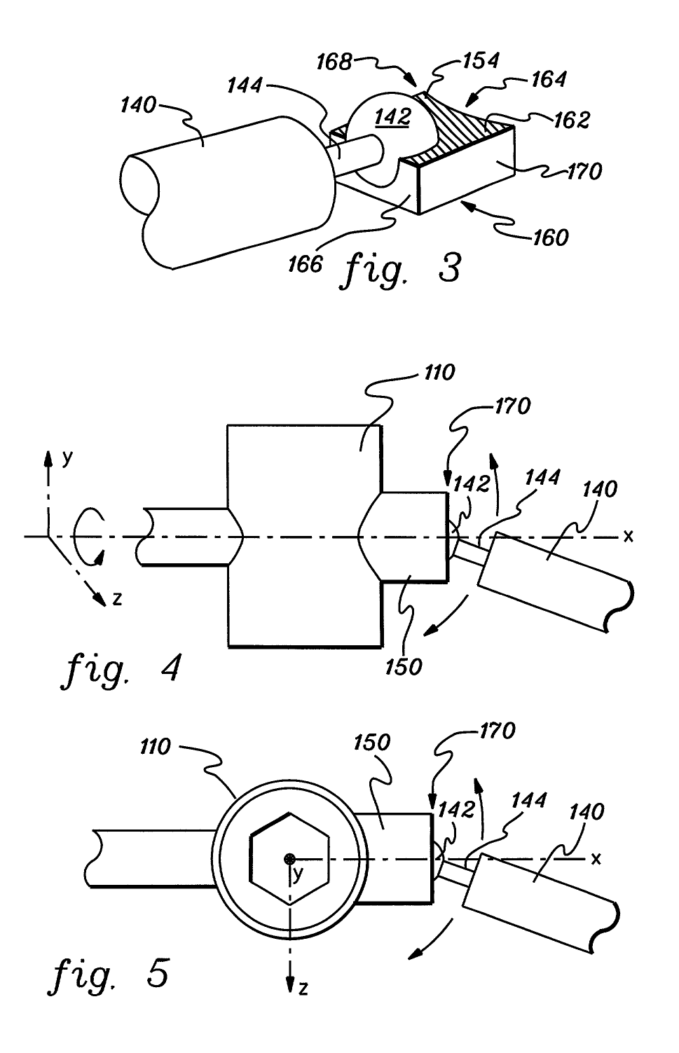

Male member 142 and the socket defined in socket housing 150 form a joint 170 which permits adjacent abutments 110, 120 to move relative to each other. The outer surface of male member 142 and the inner surface of the socket provide load bearing areas for joint 170. As illustrated in FIGS. 4 and 5, joint 170 allows movement or adjustments of the abutment in three dimensions (e.g. about the x, y, and z directions) providing a wide range of motion for adjacent abutments relative to each other. Cylindrical extension 144 of connecting segment 120 permits limited lateral motion or side to side (around the y-axis) and up and down motion (around the z-axis) of joint 170. As shown in FIG. 4, joint 170 permits 360 degree rotational movement around the x-axis.

In an alternate embodiment illustrated in FIG. 7, end wall 166 of upper and lower portions 152, 154 of socket housing 150 and the end of cylindrical shaft 140 of connecting segments 140 connecting to male member 142 may be angled away from each other or beveled to opposite degrees (e.g. 5 and −5 degrees) to form beveled surfaces 710, 720, respectively, which permit lateral and up and down movements of connecting segment 140 and socket housing 150 relative to each other. In this embodiment, cylindrical extension 144 is not required. This beveling does not effect the 360 rotation around the x-axis. FIG. 7 also illustrates an extension 750 of the implant which may extend from an implant, through the gum tissue and interface with an abutment.

With the use of joint 170 created by male member 142 and the socket formed by upper and lower portions 152, 154 of socket housing 150, the distance between adjacent abutments 110, 112 becomes the most critical measurement for fabricating the framework 100 because the angular difference required between adjacent abutments due to divergent implants may be accommodated by the moveable action of the male member and socket arrangement. Unlike a framework that is cast, the particular angular position of the abutment relative to the implant need not be exact because of the moveable (e.g. rotatable, articulatable or swivel) action of the abutment created by joint 170 which can accommodate variance in implant positioning between two abutments. Thus, the divergence of each implant does not have to be determined precisely in all cases. Also, if the implant diverges over time by altering conditions in the mouth and bones in the face (e.g. flexure of the mandible), joint 170 may adapt and reposition itself automatically without the need to manufacture an entirely new framework or frequent a dentist office.

In an alternate embodiment, the male member and corresponding hollow interior of the socket may be formed in other shapes, such as, for example, slightly spherical, oval, cylindrical or the like, so long as these shapes allow the joint to permit the abutment to move relative to the connecting segment or adjacent abutment. In yet another embodiment, the joint may be formed by inserting the connecting segment directly into a bore or recess formed in the side surface of cylindrical tube 130 of abutment 110.

In yet another embodiment, joint 170 may be configured to move abutment 110 along the x-axis relative to connecting segment 140. In this embodiment, the socket formed by socket housing 150 may be cylindrical in shape to enable male member 142 to also slide therein along the cylindrical-shaped socket.

A second segment 122 is connected between adjacent abutments 112 and 114. Similar to first segment 120, second segment 122 is connected at one end to a male member by a cylindrical extension having a smaller diameter than the cylindrical shaft. The other end of the second segment is attachable to abutment 114 by, for example, laser welding means. Abutment 112 includes a socket housing extending radially from the outer surface of the cylindrical tube forming abutment 112 towards adjacent abutment 114. The socket housing defines a socket matching the shape of the male member of the first segment. The socket housing and connecting segment 122 including male member 142 form a joint enabling abutment 112 to move (e.g. rotate, swivel, articulate and/or slide) relative to abutment 114. The main difference between connecting segments 120 and 122 may be the length of the cylindrical shaft that fits between the adjacent abutments for which they are connecting.

A third segment 124 similar to the first two segments is connected between adjacent abutments 114 and 116. Similar to first and second segments 120, 122, third segment 124 is connected at one end to a male member by a cylindrical extension having a smaller diameter than the cylindrical shaft. The other end of third segment 124 is attachable to abutment 114 by, for example, laser welding means. Abutment 116 includes a socket housing extending radially from the outer surface of the cylindrical tube forming the abutment towards adjacent abutment 114. The socket housing defines a socket matching the shape of the male member of third segment 124. The socket housing and connecting segment 122 including male member 142 form a joint enabling abutment 114 to move (e.g. rotate, swivel, articulate and/or slide) relative to abutment 116. In alternate embodiments, the socket housings for two different connecting segments may be mounted on the same abutment. Also, some of the adjacent abutments may be connected by just a cylindrical shaft extending between the abutments.

Framework assembly may also include extensions 180 extending radially from end abutments 110 and 116. These extensions 180 are used to further support dentures extending deeper into a user's mouth. Each extension 180 may include a cylindrical shaft 182 and a spherical member 184 extending radially from the side surface of cylindrical shaft 182. Spherical member 184 may, for example, connect to an O-ring embedded in the lower portion of the denture prosthesis.

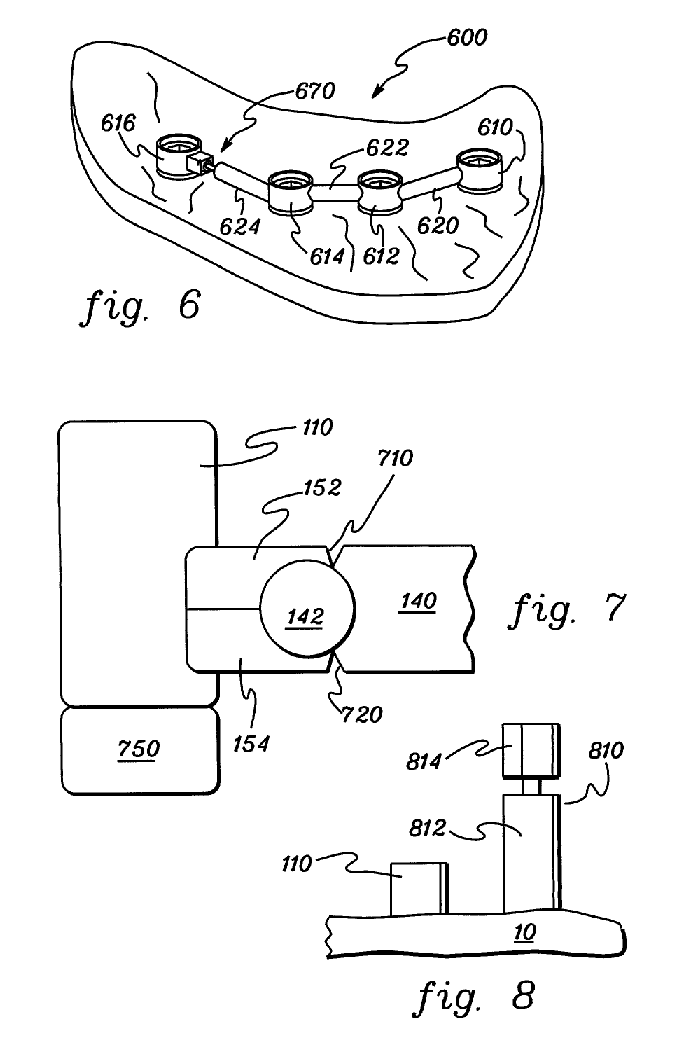

FIG. 6 illustrates another embodiment of the framework assembly in accordance with the principles of the present invention. In this embodiment, framework 600 is provided with only one joint 670 between abutments 614 and 616, while connecting segments 620, 622 extending between the other adjacent abutments 610 and 612, 612 and 614, are straight, cylindrical shafts. Both ends of cylindrical shafts 620, 622 may be laser welded to the outer cylindrical surfaces of the abutments.

One procedure for fabricating the framework for securing to divergent implants in a patient's jaw and to an overlying denture or other dental prosthesis in accordance with the principles of the present invention will now be described in more detail. Once the implants have been placed and have undergone osseointegration with the patient's jaw bone, dual-purpose impression posts are secured to the implants. As shown in FIG. 8, dual-purpose impression posts 810 include a cylindrical base 812 attached to a retention head 814. Impression posts 810, particularly cylindrical base portions 812, are selected to match the implant brand and diameter being used. Retention heads 814 are grooved or provided with protrusions extending outwardly in order to prevent impression posts 810 from moving in the impression material used to make a master mold of a patient's mouth, as will now be described.

Next, the master cast of the patient's mouth is created. An open tray impression technique, which is commonly known in the art, may be used to ensure greater accuracy of the master cast of the denture. At this point, impression material, such as, for example, polyvinyl siloxane, is injected into the patient's mouth around impression posts 810 so as to fill the impression tray. After setting, the impression tray is then removed by unscrewing impression posts 810. The master cast is intended to provide an exact replica of the patient's mouth, including the exact location of the implants, from which a stone-type (e.g. gypsum) model of the patient's mouth is made.

After the model cast is formed, retention heads 814 of the impression posts 810 and a portion of cylindrical base 812 are removed and the remaining portion of cylindrical base 812 of impression posts 810 is used as an abutment for the framework assembly to connect to the implants. Typically, the impression posts used to make the master cast are used once for the impression and then discarded. In the present invention, however, the impression posts serve the dual purpose of aiding to form the impression model and to form an abutment used in the final framework to interface with the implant.

Once the impression posts are modified to form the abutments, the connecting segments are pre-fabricated by measuring between the abutments positioned on the implants inserted into the stone-type model. The connecting segments are milled and cut to fit between the abutments.

For an abutment including a joint, lower portion 154 of socket housing 150 is first laser tacked to the outer side surface of the abutment. The settings for the laser welder used to assembly the components of the framework assembly are well known in the art and depend on the type of laser used, the power outage of the laser, the beam or spot size and diameter, the duration of the beam or spot and the frequency of the pulse, in addition to other parameters known in the art.

Male member 142 of the connecting segment is then inserted into curved recess 158 provided in inner side wall 162 and end wall 166 of lower portion 154. Upper portion 152 of socket housing 150 is then closed over the top of male member 142 to form the socket as discussed above. Upper and lower portions 152, 154 of socket housing 150 are then laser tacked in place and then seam welded together and to the abutment.

The laser welds may be reinforced using, for example, titanium allow filler wire (e.g. 0.25 millimeter). After reinforcing with laser wire, the welds may be surface blended with the laser welder. The assembled framework is then polished and finished by conventional methods to remove sharp edges and burrs that may exist.

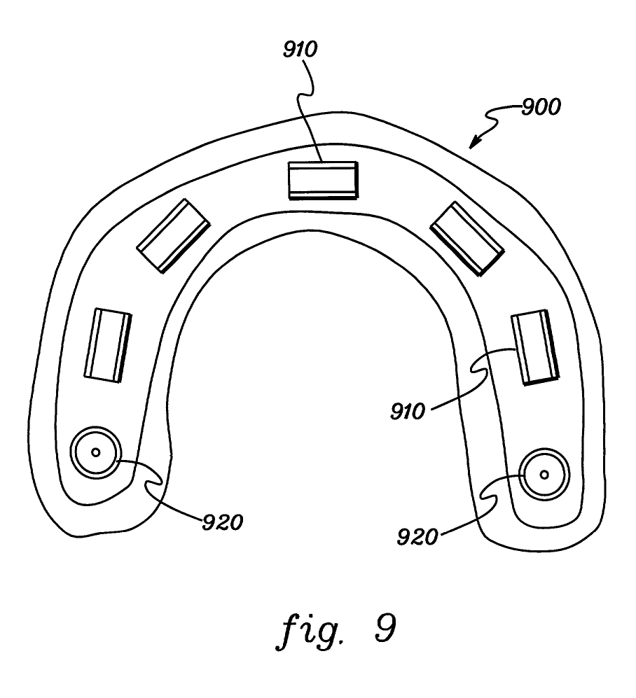

After the framework is assembled, the framework can support and retain dentures. As shown in FIG. 9, dentures 900 include titanium clips 910 which are attachable to the outer cylindrical surface of connecting segments 140 of the framework assembly. The dentures may also include nylon attachments or O-ring attachments 920 for attaching by, for example, snapping down on spherical members 184 extending upwardly from the side surface of cylindrical shaft 182 of extension 180. The nylon attachments, clips and O-ring attachments are coupled to the bottom portion of the dentures and work to transfer stress away from the implants inserted into a patient's jaw bone.

When the framework assembly is complete, it is placed into position in the patient's mouth and the retainer screws are used to secure the framework to the implants, using a hex driver tool. The hex driver tool includes a cylindrical head and shaft extending from a bottom surface the head. The shaft has an outer surface corresponding to the recess defined in the top of the retaining threaded members used to secure the framework to the implants.

The abutments are secured one at a time to the implants. With the joint(s) of the framework made in accordance with the principles of the present invention, each abutment is moveable (e.g. rotatable, swivels, articulatable and/or slidable) relative to adjacent abutments. The joint allows passive seating of multiple abutments onto existing implants, which is crucial for the long term integration between dental implants and bone. In addition, the joint enables the placement of implants on nonparallel locations and in higher quality bone.

Since the framework made in accordance with the principles of the present invention are not cast, titanium or titanium alloy is the preferred material because of its biocompatible and physical properties as well as the relatively low cost as compared to the precious metals typically used in cast frameworks. It is also highly recommended that the clips and O-ring attachments embedded in the dentures and the implants be made from titanium or titanium alloy to avoid stacking different metals on top of each other which could cause corrosion. Moreover, the use of laser welding techniques to fabricate the framework avoids any waxing, casting, finishing and soldering and eliminates the need to use expensive precious metal alloys such as palladium, platinum and gold.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the following claims.

What is claimed is:

1. A framework assembly for supporting a prosthesis and for passively fitting onto implants installed in bone, said assembly comprising:
a first abutment attachable to a first implant;
a second abutment attachable to a second implant;
said first abutment attachable to said second abutment by a joint, wherein said first abutment is movable relative to said second abutment.

2. The framework assembly of claim 1, wherein said joint comprises a male member and a housing defining a socket, the male member being moveably mounted in the socket of the housing.

3. The framework assembly of claim 2, wherein the male member comprises a shaft extending therefrom, the shaft being mounted on the first or second abutment.

4. The framework assembly of claim 3, wherein the housing is mounted on the other of the first or second abutment.

5. The framework assembly of claim 1, wherein said first abutment is articulatable relative to said second abutment.

6. The framework assembly of claim 1, wherein said first abutment is rotatable relative to said second abutment.

7. The framework assembly of claim 1, wherein said first abutment swivels relative to said second abutment.

8. The framework assembly of claim 1, wherein said first abutment slides relative to said second abutment.

9. A framework assembly for supporting a prosthesis and for passively fitting onto at least two implants embedded in bone, said framework assembly comprising:
a first abutment attachable to a first implant;
a second abutment attachable to a second implant;
a connecting segment extending between the first and second abutments, said connecting segment secured at one end to said first abutment or second abutment, the other end of the segment connected to a male member, the male member received by a socket formed in a socket housing, the socket housing secured to the other of said first abutment or said second abutment, wherein first abutment is moveable relative to said second abutment.

10. The framework assembly of claim 9, wherein said first abutment is pivotable relative to said second abutment.

11. The framework assembly of claim 9, wherein said first abutment is articulatable relative to said second abutment.

12. The framework assembly of claim 9, wherein said first abutment swivels relative to said second abutment.

13. The framework assembly of claim 9 further comprising a third abutment attachable to a third implant, said third abutment attachable to said first abutment or said second abutment by a joint, wherein said third abutment is moveable relative to said first abutment and second abutment.

14. A framework assembly for supporting a prosthesis and for passively fitting onto implants installed in a bone, said assembly comprising:
a plurality of abutments, each of said plurality of abutments attachable to a corresponding implant; and
at least one joint associated with a pair of adjacent abutments, wherein one abutment of the at least one pair of adjacent abutments including said joint is moveable relative to the other abutment of the at least one pair of adjacent abutments including said joint.

15. The assembly of claim 14, wherein said at least one joint comprises a male member and a housing defining a socket, the male member being moveably mounted in the socket of the housing.

16. A joint, said joint comprising:
a male member; and
a female member for receiving the male member, wherein the joint permits adjacent abutments used in a framework assembly for supporting prosthesis and for passively fitting to implants embedded in bone to move relative to each other.

17. The joint of claim 16, wherein said male member comprises a convex member and said female member comprises a socket formed by a socket housing, the shape of said socket corresponding to the shape of the convex member.

18. A method of making a framework assembly for attaching to implants embedded in bone and for securing implants to an overlying prosthesis, said method comprising:
taking an impression of the implant location in a patient's bone to which a prosthesis is to be attached using impression posts;
using the impression, fabricating a model of the patient's bone including implants at the implant locations;
cutting the impression posts to form abutments for the framework assembly, said abutments attachable to the implants; and
attaching by laser welding means adjacent abutments to each other by connecting segments, wherein at least one pair of adjacent abutments are attached to each other by a joint, wherein one of the abutments of the at least one pair of adjacent abutments is movable relative to the other abutment of the at least one pair of adjacent abutments.

19. The method of claim 18, wherein the joint comprises a male member connected to one end of the connecting segment, and a socket housing, the socket housing including an upper portion and a lower portion, the upper and lower portions of the socket housing having corresponding recesses that define a socket when put together, said method further comprising:
laser welding either the upper portion or the lower portion of the socket housing to one abutment of the at least one pair of adjacent abutments;
inserting the male member into the recess formed by the upper or lower portion of the socket housing laser welded to the abutment;
laser welding the other of the upper portion or lower portion of the socket housing together with the male member in the socket; and
laser welding the other end of the connecting segment opposite the male member to the other abutment of the at least one pair of adjacent abutments.
